(19) United States
(12) Patent Application Publication
(10) Pub. No.: US 2003/0163085 A1
(43) Pub. Date: Aug. 28, 2003

(54) Catheter hand-piece apparatus and method of using the same

(76) Inventors: Howard M. Tanner, Logan, UT (US); Hugh H. Trout III, Washington, DC (US); Frank Patterson, Exeter, NH (US); Terry J. Dahl, Santa Barbara, CA (US)

Correspondence Address: COLLIER SHANNON SCOTT, PLLC, 3050 K STREET, NW, SUITE 400, WASHINGTON, DC, 20007, US

(21) Appl. No.: 10/345,143

(22) Filed: Jan. 16, 2003

Related U.S. Application Data
(60) Provisional application No. 60/348,395, filed on Jan. 16, 2002.

(51) Int. Cl. A61M 31/00

(52) U.S. Cl. 604/95.01

(57) ABSTRACT

The present invention is directed to a catheter hand-piece apparatus for use in surgical procedures to control the positioning and functioning of a catheter assembly. The catheter hand-piece apparatus includes a handle, which is connected to a catheter assembly, and a fastener module located within the handle. In accordance with an embodiment of the present invention, the handle comprises a clear molded lid that is pivotally attached between two molded enclosures, a tubular axle located between the enclosure sections and attached thereto by a snap ring, and a nosepiece, which is attached to a catheter sheath of the catheter assembly. The interchangeable fastener module may be replaced mid-procedure and includes an irrigation port and a flexible sheath, which is attached to the port and has a tubular adjustment knob slidably positioned about it. The knob may be interchangeably mounted to the handle. The module further includes a sheath-protected optical fiber for selectively ablating an area within a vessel and a fastener-pusher. Two o-rings are included for creating fluid-tight seals in the module. Furthermore, at least one fastening means is loaded over, within, or in conjunction with the optical fiber of the fastener module to be deployed at the surgical site.

### CROSS-REFERENCE TO RELATED APPLICATIONS

The present inventions relate to, and are entitled to the benefit of the earlier filing date and priority of, U.S. Provisional Patent Application 60/348,395, filed Jan. 16, 2002.

### FIELD OF THE INVENTION

The present invention relates to a catheter hand-piece apparatus. In particular, the present invention is directed to a catheter hand-piece apparatus for use in surgical procedures to control the positioning and functioning of a catheter assembly.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and, death.

Aortic aneurysms are the most common form of arterial aneurysm and are life threatening. The aorta is the main artery, which supplies blood to the circulatory system. The aorta arises from the left ventricle of the heart, passes upward and bends over behind the heart, and passes down through the thorax and abdomen. Among other arterial vessels branching off the aorta along its path, the renal arteries supply blood to the kidneys. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in the abdominal portion of the aorta between the renal and iliac arteries. This portion is particularly susceptible to weakening, resulting in an aortic aneurysm. Such an aneurysm is often located near the iliac arteries. An abdominal aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because presently no statistical benefit exists in performing such procedures.

Aneurysms in the abdominal aorta are associated with a particularly high mortality rate; accordingly, current medical standards call for urgent operative repair. Abdominal surgery however, results in substantial stress to the body. Although the mortality rate for an aortic aneurysm is extremely high, there is also considerable mortality and morbidity associated with open surgical intervention to repair an aortic aneurysm. This intervention involves penetrating the abdominal aorta to the level of the aneurysm to reinforce or replace the diseased section of the aortic wall. A prosthetic device, typically a synthetic tube graft, is used for this purpose. The graft serves to exclude the aneurysm from the circulatory system, thus relieving pressure and stress on the weakened aneurismal section of the aorta.

Repair of an aortic aneurysm by surgical means is a major operative procedure. Substantial morbidity accompanies the procedure, resulting in a protracted recovery period. Furthermore, the procedure entails a substantial risk of mortality. While surgical intervention may be indicated, the procedure carries attendant risk, as certain patients may not be able to tolerate the stress of intra-abdominal surgery. It is, therefore, desirable to reduce the mortality and morbidity associated with intra-abdominal surgical intervention.

In recent years, methods have been developed in an attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

Although in recent years certain techniques have been developed that may reduce the stress, morbidity, and risk of mortality associated with surgical intervention to repair aortic aneurysms, including delivery catheter assemblies, Applicants are not aware of any system that provides a hand-piece apparatus for effectively controlling the positioning and functioning of the catheter assembly. In particular, none of the prior art devices incorporate a catheter assembly attached to the hand-piece outside of the body that includes controls for positioning the catheter adjacent to the surgical site and a fastener module for positioning and delivering a fastening means to the surgical site. Additionally, the prior art neither provides an apparatus that indicates the number of fastening means already deployed nor fasteners remaining in the module. Further, none of the prior art references provide a device for replenishing fastening means mid-procedure without having to remove and reinsert the entire catheter assembly. Moreover, the prior art does not provide a device that can complete such tasks in a reliable and repeatable manner. In light of these concerns, the present invention is directed to an apparatus that controls positioning and functioning of an attached catheter assembly. The components of the hand-piece provide the reliable and repeatable performance of all aforementioned tasks during a surgical procedure.

It is an advantage of an embodiment of the present invention to provide an apparatus for remotely controlling the positioning and functioning of a catheter assembly during a surgical procedure.

It is another advantage of an embodiment of the present invention to provide an apparatus for controlling the positioning and functioning of a catheter assembly during a surgical procedure.

It is another advantage of an embodiment of the present invention to provide an apparatus for controlling the delivery of at least one fastening means to and deployment at a surgical site.

It is another advantage of an embodiment of the present invention to provide an apparatus for controlling a catheter's advancement within a vessel to a position adjacent to a surgical site.

It is another advantage of an embodiment of the present invention to provide an apparatus for controlling the articulation or realignment of the catheter tip with respect to the catheter's longitudinal axis.

It is another advantage of an embodiment of the present invention to provide an apparatus for controlling the advancement or withdrawal of an inner sheath and optical fiber of a catheter assembly.

It is another advantage of an embodiment of the present invention to provide an apparatus for locking or unlocking the positioning of an articulated catheter tip or advanced inner sheath of a catheter assembly.

It is another advantage of an embodiment of the present invention to provide an apparatus for adjusting the radial positioning of an advanced inner sheath of a catheter assembly.

It is another advantage of an embodiment of the present invention to provide an apparatus for adjusting an optical fiber tip's axial positioning to influence deployment accuracy.

It is another advantage of an embodiment of the present invention to provide an apparatus for advancing an optical fiber, at least one fastening means, and a fastener-pusher.

It is another advantage of an embodiment of the present invention to provide a remote apparatus that enables the independent withdrawal of an optical fiber from within the advanced fastener pusher and fastener means.

It is another advantage of an embodiment of the present invention to provide an apparatus that indicates the number of fastening means deployed or remaining within a fastener module.

It is another advantage of an embodiment of the present invention to provide an apparatus to replenish the catheter with a supply of fastening means mid-procedure without having to remove and reinsert the entire catheter assembly.

It is another advantage of an embodiment of the present invention to provide a procedure for positioning and locking a delivery catheter assembly within a vessel in preparation for deployment of at least one fastening means at a surgical site.

It is another advantage of an embodiment of the present invention to provide a procedure for controlling deployment of at least one fastening means at a surgical site and thereafter determining and/or displaying the quantity of fastening means deployed.

It is another advantage of an embodiment of the present invention to provide a procedure for repeatedly repositioning a catheter assembly within a vessel during a surgical procedure.

Additional advantages of the invention are set forth, in part, in the description which follows and, in part, will be apparent to one of ordinary skill in the art from the description and/or from the practice of the invention.

### SUMMARY OF THE INVENTION

Responsive to the foregoing challenges, Applicant has developed an innovative catheter hand-piece apparatus for controlling functions of a catheter during a surgical procedure. An embodiment of the catheter hand-piece apparatus includes a handle, which is connected to a catheter assembly, and a fastener module, which is permanently or interchangeably located within the handle.

In accordance with an embodiment of the present invention, the handle includes a clear molded lid, pivotally attached between two molded enclosure sections, a tubular axle located between the enclosure sections and attached thereto by a snap-ring, and a nosepiece, which is adjustably mounted to the axle and attached to a catheter sheath of the catheter assembly. The nosepiece may be spring-loaded or fixed. The fastener module includes an irrigation port and a flexible sheath attached thereto that extends to a micro-adjustment boss component to which it is also attached. A tubular micro-adjustment knob is slidably positioned about the flexible sheath, thread-assembled to the micro-adjustment boss component and, interchangeably mounted to the handle. The module also has a sheath-protected optical fiber, enabling transmission of laser energy to a surgical site and which extends from the irrigation port to a connector that is slidably positioned thereon. A tubular fastener-pusher further surrounds the optical fiber and extends from the irrigation port to a point abutting the most proximally over-fiber positioned fastening means. The fastener-pusher may be comprised of polyimide tubing, an extension spring, or any other suitable material. An internal o-ring located within the micro-adjustment boss component creates a fluid-tight seal between itself and the outside surface of the fastener-pusher. An external o-ring positioned about the fastener-pusher at its proximal end creates a fluid-tight seal between itself and the flexible sheath. Further, the module includes at least one fastening means, which is loaded over, within, or in conjunction with the optical fiber, wherein the optical fiber is independently movable with respect to the fastening means. The fastening means may be comprised of nitinol or stainless steel or any other mechanically similar, biologically appropriate material.

The present invention may also include a fastener module control mechanism for advancing and withdrawing components of the fastener module. The fastener module control mechanism is also located within the handle. The fastener module control mechanism has an adjustment knob pair externally positioned on opposite sides of the molded enclosure sections. The knob pair is mounted to a geared shaft located within the handle which, through rack and pinion detailing, propels the fastener module distally. The mechanism also includes a pair of advancing arms. The first arm is pivotally mounted to a carriage component, which is located within the molded enclosures, and slidably attached to the lid. The second arm is slidably located within the carriage component and between the handle's enclosure sections. The arms advance the fastener module when in association with the knob pair and gear component. The proximal end of the second advancing arm may work collaboratively with the micro-adjustment boss component to facilitate linear micro-adjustment of the optical fiber's distal positioning.

An embodiment of the present invention may further include an articulation control mechanism incorporating a flexible filament to adjust the position of the catheter tip during a surgical procedure. This mechanism is located within the handle and attached to the free end of the catheter's filament. The mechanism includes an adjustment knob pair, externally positioned on opposite sides of the enclosure sections and mounted,to a gear component, which is located within the handle. The gear component translates rotary motion of the knob pair, through rack and pinion detailing, into linear motion of a tubular mounting collar and an associated boss, thereby pulling a adjustment filament attached thereto.

In accordance with an embodiment of the present invention, the catheter hand-piece apparatus may also include an apposition control mechanism, which is located within the handle. This mechanism advances an inner sheath with respect to the outer sheath of the catheter assembly, thereby creating an appositional force between itself and a graft and vessel wall combination. The mechanism includes an adjustment knob pair, which is externally positioned on opposite sides of the enclosure sections. The knob pair is mounted to a gear component, which is located within the handle, and adjusts the angular positioning of a carriage component through rack and pinion detailing. The carriage component is attached to an inner sheath of the catheter assembly, thereby advancing the inner sheath of the catheter assembly for creating an appositional force between the sheath and a graft and vessel wall combination.

An embodiment of the present invention may further include a locking mechanism, which is located within the handle, for selectively engaging the articulation or apposition control mechanism. The locking mechanism comprises a spring-loaded assembly of three components, located within the handle. The components include an axle-mounted flexible yoke component working in collaboration with an activation button and ratchet detail within both articulation and apposition mechanism gear components. Depression of the button allows internal cam detailing within the yoke to collapse into a groove feature within the button shaft, thereby reducing the ratchet holding force between the external yoke detailing and the articulation and apposition mechanism gear components and thereafter, adjustment of those same control mechanisms.

In an alternative embodiment of the present invention, the catheter hand-piece apparatus includes one tubular handle, which is connected to a catheter assembly, and a second tubular handle containing a fastener module, which engages with the first handle. The first handle may include an articulation control mechanism. This mechanism is attached to a adjustment filament to change the angular position of the catheter during a surgical procedure. The handle may also include an apposition control mechanism. This mechanism extends the inner sheath distally beyond the outer sheath of a catheter assembly. The inner sheath contacts the graft and vessel wall; continued extension forces the outer sheath against the opposing wall of the vessel, thereby creating an appositional force between the outer sheath and the opposing graft and vessel walls. An irrigation port may further be included in the handle, providing for delivery of an irrigation solution around at least one fastening means. The solution may be heparinized.

The articulation control mechanism and the apposition control mechanism may each comprise a knob, which surrounds the first handle, mounted to a gear component located therein, for translating rotary into linear motion. Alternatively, each mechanism may comprise a linear slide and a locking mechanism for achieving linear motion.

The fastener module may include a knob, which surrounds the second handle, mounted to a gear component located therein, for translating rotary into linear motion. The linear motion extends an optical fiber, which is attached to the second handle, thereby deploying at least one fastening means over the optical fiber. In addition, the fastener module may include a second knob surrounding the second handle. The knob is mounted to a second gear component located therein, for translating rotary into linear motion, thereby adjusting the length of the optical fiber. Alternatively, the fastener module may include linear slides and locking mechanisms. The first linear slide pushes an optical fiber that is attached to the second handle thereby deploying at least one fastening means over the optical fiber. The second linear slide adjusts the length of the optical fiber. The locking mechanisms secure the position of the linear slides.

In another embodiment of the present invention, the catheter hand-piece apparatus includes a single tubular handle, which is connected to a catheter assembly. It also includes an articulation control mechanism, located therein, which is attached to a adjustment filament, for adjusting the angular position of the catheter during a surgical procedure. An apposition control mechanism, which is located therein, is further included for advancing an inner sheath distally beyond an outer sheath of a catheter delivery assembly. As the inner sheath contacts the graft and vessel wall, its continued extension forces the outer sheath against the opposing wall of the vessel, thereby creating an appositional force between the outer sheath and the opposing graft and vessel wall. The apparatus also includes a fastener mechanism, located within the handle, for linearly adjusting the free length of an optical fiber, thereby deploying at least one fastening means over, within, or in conjunction with the optical fiber, and an irrigation port, within the handle, for delivery of an irrigation solution, that may be heparinized, around at least one fastening means.

The articulation control mechanism and the apposition control mechanism each may include a knob surrounding the handle. The knobs are mounted to gear components, which are located within the handle, for translating rotary into linear motion. Alternatively, each mechanism may comprise a linear slide and associated locking mechanism for achieving linear motion.

The fastener mechanism may include a first knob, which surrounds the handle, mounted to a gear component within the handle, for translating rotary into linear motion. The linear motion pushes an optical fiber, which is attached to the handle, thereby deploying at least one fastening means over, within, or in conjunction with the optical fiber. In addition, the fastener mechanism may include a second knob surrounding the handle. The second knob is mounted to a second gear component located within the handle, for translating rotary into linear motion, thereby adjusting the length of the optical fiber. Alternatively, the fastener mechanism may include linear slides and locking mechanisms. The first linear slide pushes an optical fiber that is attached to the handle thereby deploying at least one fastening means over, within, or in conjunction with the optical fiber. The second linear slide adjusts the length of the optical fiber. The locking mechanisms secure the position of the linear slides.

In yet another alternative embodiment of the present invention, the catheter hand-piece apparatus includes a first handle assembly, which is attached to an outer catheter of a catheter assembly, and a fastener module, which, as a second handle assembly, is interchangeably attached to the first handle assembly. The first handle assembly includes a steering housing, a steering ring or sleeve surrounding the housing for articulating the outer catheter, a steering ring or sleeve lock located circumferentially about the housing adjacent to the steering ring or sleeve, depression of which allows free rotation of the steering ring or sleeve, and a hemostasis port within the housing, which is attached to a proximal end of the outer catheter. The terms ring and sleeve will be used interchangeably throughout the application. The fastener module includes a deployment housing, a hemostasis port within the housing, which is attached to an inner catheter of the catheter assembly, an advancement slide within the housing, which is attached to the hemostasis port, an advancement sleeve surrounding the housing for actuating the advancement slide, an advancement sleeve lock located on an outer surface of the housing adjacent to the advancement sleeve, a deployment sleeve surrounding the housing, which couples a deployment slide to the advancement slide, and a tab located on the outer surface of the housing adjacent to the deployment sleeve to prevent its inadvertent rotation. The fastener module may further include deployment housing quick release detailing and a fiber position micro-adjustment. The handle assemblies may also include at least one locating tab and corresponding slot.

According to the present invention, embodiments of the catheter hand-piece apparatus may further include a cradle for stabilizing the apparatus during a surgical procedure.

The present invention is also directed to a method of positioning a catheter assembly within a vessel in preparation for deployment of at least one fastening means at a surgical site, comprising the steps of controlling the advancement of the catheter assembly to a position adjacent the surgical site, articulating a catheter tip of the catheter assembly by adjusting a adjustment filament embedded therein, which is attached to an articulation control mechanism located within the catheter hand-piece, advancing an inner sheath of the catheter assembly with an apposition control mechanism located within the hand-piece until the distal end of the inner sheath makes contact with a graft and which upon further advancement, forces the catheter assembly into contact with a vessel wall directly adjacent to the surgical site, and thereafter locking the inner sheath in a position with a locking mechanism.

The present invention is also directed to a method for controlling deployment of a fastening means at a surgical site and determining a quantity of fastening means deployed with a catheter hand-piece apparatus, comprising the steps of creating an aperture in a graft and vessel combination, advancing an optical fiber, at least one fastening means, and a fastener-pusher with a fastener module control mechanism located within the catheter hand-piece, irrigating contact surfaces between the optical fiber and the at least one fastening means, and between the at least one fastening means and the inner surface of an inner sheath of a catheter assembly through an irrigation port located within or remotely from the catheter hand-piece, withdrawing the optical fiber independently of the at least one fastening means and the fastener-pusher with the fastener module control mechanism, and indicating the quantity of fastening means deployed or remaining in the fastener module within the catheter hand-piece.

The present invention is also directed to a method for repositioning a catheter assembly and replenishing at least one fastener module during a surgical procedure for repeated deployment of fastening means, comprising the steps of withdrawing an inner sheath of the catheter assembly and an optical fiber with a catheter hand-piece apparatus, unlocking an articulated catheter tip and returning the tip to a collinear alignment with respect to the catheter assembly with the catheter hand-piece apparatus, repeatedly repositioning the catheter assembly to facilitate further deployment of fastening means with the catheter hand-piece apparatus, and replenishing a supply of fastening means to a surgical site mid-procedure by removing the spent fastener module and replacing it with another fastener module having at least one fastening means therein.

It is to be understood that both the foregoing general description and the following detailed description are exemplary and explanatory only, and are not restrictive of the invention, as claimed. The accompanying drawings, which are incorporated herein by reference, and which constitute a part of this specification, illustrate certain embodiments of the invention, and together with the detailed description serve to explain the principles of the present invention.

### BRIEF DESCRIPTION OF THE DRAWINGS

In order to assist the understanding of this invention, reference will now be made to the appended drawings, in which like reference numerals refer to like elements. The drawings are exemplary only, and should not be construed as limiting the invention.

FIG. 1 is an exploded view of a catheter hand-piece apparatus in accordance with an embodiment of the present invention,

FIG. 2 is a schematic illustration of an alternative embodiment of a catheter hand-piece apparatus in accordance with the present invention;

FIG. 3 is a schematic illustration of another embodiment of the catheter hand-piece apparatus in accordance with the present invention;

FIG. 4A and B are sectional views of yet another embodiment of the catheter hand-piece apparatus; and

FIG. 5 illustrates a cradle for use with the catheter hand-piece apparatus illustrated in FIG. 1, FIG. 2, FIG. 3, and FIG. 4.

### DETAILED DESCRIPTION OF THE INVENTION

The following description of an embodiment of the present invention is described, for purpose of example, in connection with the repair of an aortic aneurysm. It is contemplated that the embodiments described herein are capable of use in the repair of other vessels and in other procedures. Thus, it is intended that the present invention cover the modifications and variations of the invention, provided they come within the scope of the appended claims and their equivalents.

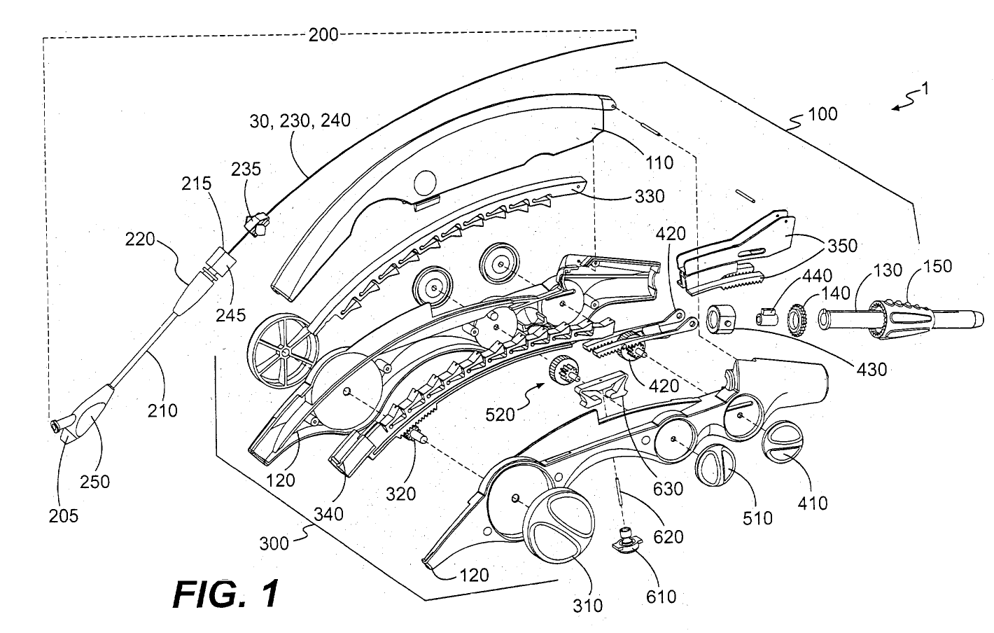

The catheter hand-piece apparatus will now be described in connection with FIGS. 1-5. As illustrated in FIG. 1, an embodiment of the catheter hand-piece apparatus 1 includes a handle 100 and a fastener module 200 interchangeably located within the handle.

The handle 100 preferably consists of two molded enclosure sections 120 to which a clear molded lid 110 is pivotally connected. A tubular axle 130 is located between the enclosure sections and attached thereto by a snap-ring 140. An optionally spring-loaded nosepiece 150 is adjustably mounted to the axle. The nosepiece is connected to the catheter sheath.

As depicted in FIG. 1, the fastener module 200 preferably contains an irrigation port 205 for irrigating the contact surfaces between, an optical fiber and the internal surface of at least one fastening means, and the contact surfaces between the external surface of the at least one fastening means and the inner wall of the inner sheath of a catheter assembly during deployment of at least one fastening means. The irrigation port may incorporate Luer-lock, friction fit, bayonet fit, or any similar detailing. A flexible sheath 210 preferably extends from the irrigation port 205 to a micro-adjustment boss component 215. The flexible sheath 210 may comprise, but is not limited to: thermoplastic, PVC, polyurethane, Pebax®, or any other suitable material. A tubular micro-adjustment knob 220 is slidably positioned about the flexible sheath 210 and thread assembled to the micro-adjustment boss component 215. The knob 220 may be interchangeably mounted to the handle 100 of the catheter hand-piece.

The fastener module 200 preferably also includes an optical fiber 30, enabling transmission of laser energy to a surgical site. The fiber 30 is surrounded by a protective sheath 230, which extends from the irrigation port to a connector component 235. A tubular fastener-pusher 240 also surrounds the fiber 30. It extends from the irrigation port 205 to a point abutting the most proximally positioned of the, at least one fastening means loaded over or in communication with the optical fiber 30, wherein the optical fiber is independently movable with respect to the fastening means. The fastener pusher 240 may be comprised of polyimide tubing, steel wound coil, PTFE (polytetrafluoroethylene), FEP (fluorinated ethylenepropylene), or any other suitable material. The fastening means may be comprised of nitinol or stainless steel or any other mechanically similar, biologically appropriate material. There are also two o-rings 245, 250 located within the fastener module 200, which create a sliding, fluid tight association between the fastener pusher 240 and the components with which it is in communication. The first o-ring 245 is internally located within the micro-adjustment boss component 215 and creates a fluid-tight seal between the o-ring 245 and the outside surface of the fastener pusher 240. The second o-ring 250 is externally positioned about the fastener pusher 240 at the proximal end and creates a fluid-tight seal between the o-ring 250 and the flexible sheath 210.

The catheter hand-piece may further comprise a fastener module control mechanism 300 for advancing and withdrawing the related components of the fastener module 200, as shown in FIG. 1. An adjustment knob pair 310 is positioned on opposite sides of the enclosure sections 120 of the handle 100. The knob pair 310 is mounted to a geared shaft 320, which is located inside the handle 100. The knob pair 310 and geared shaft 320 translate rotary into linear motion through rack and pinion detailing. The control mechanism 300 also has a pair of advancing arms 330, 340. One of the arms 330 is pivotally mounted to a carriage component 350, which is located within the handle 100, and slidably attached to the lid 110 of the handle 100. The second arm 340 is slidably located within the carriage component 350 and between the molded enclosures 120. The second arm 340 associates with the geared shaft 320 through rack and pinion detailing. The knobs 330, 340 are rotated and collaborate to advance the fastener module 200. The proximal end of the second advancing arm 340 may work collaboratively with adjustment boss 215, facilitating linear micro-adjustment of optical fiber positioning.

An articulation control mechanism 400 may also be included in the catheter hand-piece, as depicted in FIG. 1. This mechanism articulates and adjustably locks a catheter tip within the vessel to a procedure-determined angle of up to 100 degrees with respect to the catheter's longitudinal axis. An adjustment knob pair 410 is externally positioned on opposite sides of the enclosure sections 120 of the handle 100. The knob pair 410 is mounted to a geared shaft 420, which is located inside the handle 100. The knob pair 410 and geared shaft 420 translate rotary motion into linear motion through rack and pinion detailing. The rotary motion is translated into linear motion of a tubular mounting collar 430 and associated boss 440, which are connected to an indwelling catheter adjustment filament. The catheter adjustment filament may be a braided wire, a single wire, or a monofilament. A monofilament may comprise, but is not limited to: Kevlar®, Spectra®, or any other suitable material. The motion pulls the adjustment filament, thereby creating catheter articulation, or releases the filament to return the catheter to its linear position.

An apposition control mechanism 500 may also be included in the catheter hand-piece for creating an appositional force between the inner sheath of a catheter delivery apparatus and a graft and vessel wall combination. An adjustment knob pair 510 is externally positioned on opposite sides of the enclosure sections 120 of the handle 100. The knob pair 510 is mounted to a geared shaft 520, which is located inside the handle 100. The knob pair 510 and geared shaft 520 translate rotary motion into linear motion through rack and pinion detailing. This motion adjusts the angular positioning of the carriage component 350, which is attached to the inner sheath of the catheter assembly. The inner sheath is thus advanced until such action forces the catheter into contact with the graft and vessel wall directly opposite the treatment site. The carriage component 350 preferably works collaboratively with the fastener module control mechanism to ensure a constant co-planar relationship between the distal ends of the optical fiber and the inner sheath of the catheter.

The articulation control mechanism 400 and the apposition control mechanism 500 may be selectively engaged through the use of a locking mechanism 600. This mechanism is located within the handle 100 of the catheter hand-piece and consists of a spring-loaded assembly of three components 610, 620, 630, as shown in FIG. 1. The components include an axle 620 mounted flexible yoke component 630 working in collaboration with an activation button 610 and ratchet detailing of both the articulation and apposition mechanism gear components 420, 520. Depression of button 610, which may be located on the external surface of the enclosure sections 120, allows internal cam detailing within the yoke to collapse into a groove feature within the button shaft, thereby reducing the ratchet holding force between the external yoke detailing and the articulation and apposition mechanism gear components and thereafter adjustment of those same control mechanisms.

The operation of the catheter hand-piece apparatus 1 will now be described. Upon insertion of a catheter into the body, the catheter assembly preferably is positioned at the surgical site through use of the different controls on the catheter hand-piece 1. The catheter assembly preferably is connected to the catheter hand-piece 1, which remains outside the body. The catheter sheath may be rotated independently of the catheter hand-piece 1 to un-encumber its initial placement within the patient. The advancement of the catheter assembly preferably is controlled through movement of the hand-piece 1 to a position adjacent to the surgical site. Movement of the catheter directly mimics movement of the catheter hand-piece. The catheter tip preferably is articulated into a procedure-determined location and adjustably locked at an angle of up to 100 degrees with respect to the catheter's longitudinal axis. This tip articulation preferably is produced by pulling an imbedded catheter articulation filament within the catheter hand-piece 1. The adjustment filament may be a braided wire, a single wire, a monofilament or any other suitable material.

In particular, articulation control mechanism's knob pair 410 is rotated clockwise. This rotary motion is translated into linear motion of a tubular mounting collar 430 and associated boss 440 by a gear component 420 within the handle through rack and pinion detailing. Because the adjustment filament is attached to the boss 440, the adjustment filament is pulled linearly thereby creating articulation of the catheter tip. The inner sheath of the catheter assembly is then advanced through the articulated catheter with an apposition control mechanism 500 located within the catheter hand-piece 1. The apposition control mechanism's knob pair 510 is rotated counterclockwise and an associated gear component 520 within the handle transfers the rotary motion into linear motion of a carriage component 340 through rack and pinion detailing. The inner sheath is advanced by the linear the carriage 340 to which it is attached. The inner sheath is advanced until its distal end makes contact with a graft and then advanced further until it forces the catheter into contact with the vessel wall directly opposite the surgical site. The inner sheath may be locked into this position, or apposition, directly opposite the treatment site by a locking mechanism 600 located within the handle. Activation button 610 of locking mechanism 600 preferably is released, which pushes the flexible yoke 630 outward, thereby increasing the ratchet holding force between external yoke detailing and the apposition control mechanism.

The catheter hand-piece apparatus 1 also operates to control deployment of at least one fastening means from a fastener module 200 at a surgical site and to indicate the quantity of fastening means deployed. An optical fiber tip's 30 axial positioning with respect to the distal face of the most distally located over-fiber fastening means may be adjusted by the catheter hand-piece 1 micro-adjustment knob 220 to influence deployment accuracy. An aperture preferably is created in a graft and vessel combination. Laser energy transmission, or any other suitable energy transmission to create an aperture, may be activated or terminated using a remote controller. Alternatively, an energy activation control may be incorporated into the catheter hand-piece. The aperture may also be created by any other suitable means.

Following creation of an aperture, the fastener module control mechanism's knob pair 310, which is externally positioned on opposite sides of the enclosure sections 120, preferably is rotated clockwise. Inside the handle 100, the rotary motion of the knob pair 310 is translated into linear motion of two advancing arms 330, 340 by a geared shaft 320 upon which the knob pair 310 is mounted, which communicates with the second advancing arm 340 through rack and pinion detailing. The linear motion of arm 340 advances the optical fiber 30, at least one fastening means, and the fastener-pusher 240, which are part of the fastener module 200 located within the handle 100 by a relationship between arms 330, 340 and connector component 235 of the fastener module 200. The optical fiber 30 preferably is withdrawn independently of the at least one fastening means and the fastener-pusher 240 by counter-clockwise rotation of the fastener module control mechanism's knob pair 310, thereby deploying at least one fastening means. Irrigation preferably is supplied to all contact surfaces by an irrigation port 205 located within the handle 100. Irrigation may be helpful in preventing clotting, following tissue/graft ablation. The quantity of fastening means deployed or remaining in the fastener module 200 is preferably determined by viewing the connector 235 positioning with respect to the two enclosure sections. The quantity of fastening means deployed or remaining may also be determined or displayed by any other suitable means.

The catheter hand-piece 1 also operates to control catheter repositioning and fastening means replenishment within the vessel during a surgical procedure. The inner sheath of the catheter may be unlocked by disengaging the locking mechanism. The interdependent inner sheath and optical fiber 30 preferably are withdrawn within the catheter. The inner sheath is withdrawn by clockwise rotation of the apposition control mechanism's knob pair 510. The optical fiber 30 is withdrawn by counterclockwise rotation of the fastener module control mechanism's knob pair 510. The articulated catheter tip preferably is unlocked by disengaging the locking mechanism 600. The tip is returned to a collinear alignment with respect to the catheter body by rotation of the articulation control mechanism's knob pair 410. The catheter assembly preferably is repeatedly positioned and repositioned at the surgical site by realigning the catheter hand-piece 1 to move the catheter and by rotation of the different knob pairs 310, 410, 510. The repositioning of the catheter facilitates further deployment of at least one fastening means at the surgical site until the specific procedure is completed. The supply of fastening means is preferably replenished mid-procedure without necessitating the removal and replacement of the entire catheter delivery assembly by releasing and rotating the latched lid 110 positioned between the two enclosure sections 120, removing the spent fastener module 200, and replacing it with a new fastener module.

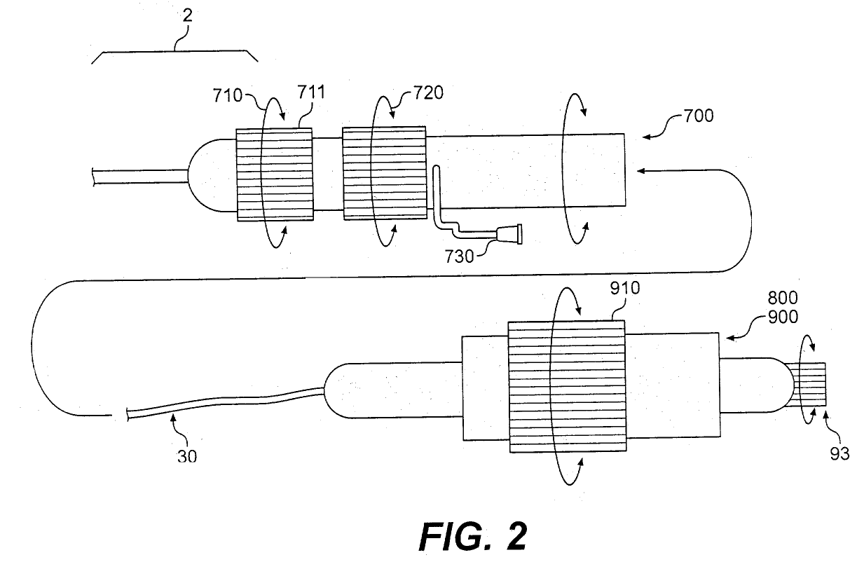

According to another embodiment of the present invention, depicted in FIG. 2, the catheter hand-piece apparatus 2 comprises two tubular handles 700, 800 and a fastener module 900. The first tubular handle 700, which is connected to a catheter assembly, includes an articulation control mechanism 710, an apposition control mechanism 720, and an irrigation port 730. The second tubular handle 800, which engages with the first tubular handle 700, includes the fastener module 900. According to this modular approach, the catheter, which is connected to the first handle 700, remains in the body while the second handle 800 can be selected to suit the specific quantity or type of fastening means needed for the surgical procedure.

The first tubular handle 700 contains the articulation control mechanism 710. A adjustment filament is attached to the mechanism 710 for creating articulation of the catheter tip. In an embodiment, the articulation control mechanism 710 comprises a knob 711 surrounding the tubular handle 700 and a gear component within the handle, which translates the knob's rotary motion into linear motion. This motion pulls the adjustment filament, thereby creating catheter articulation. In another embodiment, the mechanism 710 comprises a linear slide located externally on the tubular handle 700 and a locking mechanism that secures the position after the linear adjustment is made.

The apposition control mechanism 720 preferably is also located in the first tubular handle 700. It is connected to an inner sheath of the catheter assembly for extending the inner sheath from the outer sheath, thereby creating an appositional force between the outer sheath and a graft and vessel wall combination. This mechanism 720 advances the inner sheath of the catheter assembly through linear motion created by either of the two knobs and gear or linear slide alternatives previously discussed for the articulation control mechanism. In addition, an irrigation port 730 is located on the first handle 700 to provide for delivery of a flush solution that may be heparinized around at least one fastening means.

The second tubular handle 800 preferably comprises the fastener module 900. An embodiment of the fastener module 900 comprises a first knob 910 surrounding the handle 800 mounted to a first gear component within the handle for translating rotary into linear motion. Upon clockwise rotation of knob 910, the linear motion advances an optical fiber 30, which is attached to the handle 800, and at least one fastening means, which is located over, within, or in conjunction with the optical fiber 30. Upon counter-clockwise rotation of knob 910, the linear motion retracts the optical fiber 30, thereby deploying at least one fastening means at a surgical site. A second knob 930 and associated gear component transfers rotary into linear motion for adjusting the length of the optical fiber 30. For instance, the fiber 30 may be adjusted during packaging, sterilization, and/or actual use. An alternative embodiment of the fastener module 900 uses linear slides in place of each of the knob and gear components to achieve the linear motion desired in each case. This embodiment also includes locking mechanisms to secure the position after the linear adjustment is made.

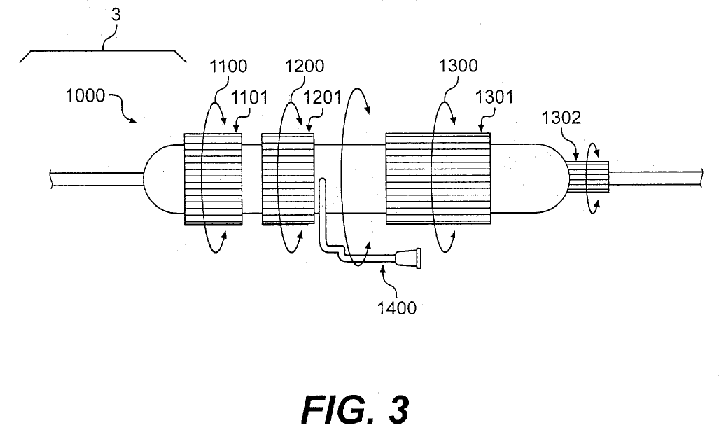

In another embodiment of the present invention, depicted in FIG. 3, the catheter hand-piece apparatus 3 comprises a single tubular handle 1000, which is connected to a catheter assembly, and an articulation control mechanism 1100, an apposition control mechanism 1200, a fastener mechanism 1300, and an irrigation port 1400 all located within the single tubular handle 1000. The articulation control mechanism 1100, apposition control mechanism 1200, and fastener mechanism 1300 all include two alternative embodiments. Each mechanism comprises at least one knob 1101, 1201, 1301, and 1302 respectively, surrounding the handle 1000 and an associated gear or at least one linear slide and locking mechanism. Both embodiments achieve linear motion. For the articulation control mechanism 1100, the linear motion pulls and releases an attached catheter adjustment filament. In the case of the apposition control mechanism 1200, the linear motion advances an inner sheath of a catheter assembly. The fastener mechanism 1300 comprises two knobs 1301, 1302 each mounted to an associated gear component or alternatively linear slides with locking mechanisms. One component advances the optical fiber 1303 and at least one fastening means and the other extends the length of the optical fiber 1303.

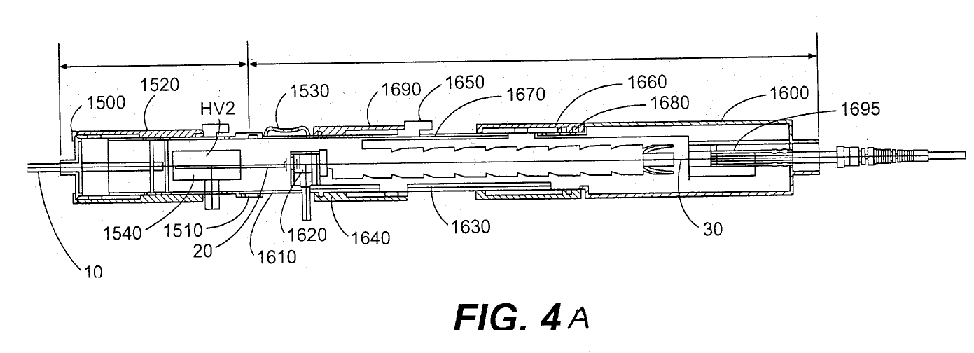

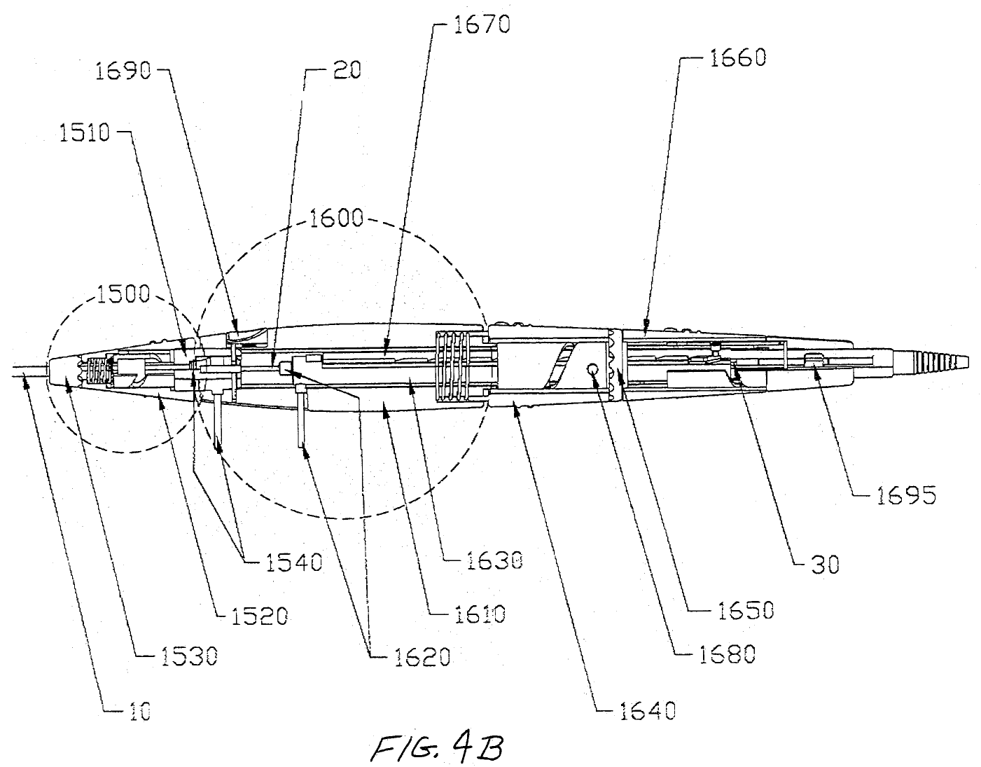

Another alternative embodiment of the catheter hand-piece apparatus is depicted in FIGS. 4A and B. This embodiment of the apparatus 4 comprises a first handle assembly 1500, which is attached to an outer catheter 10 of a catheter assembly, and a fastener module, which is a second handle assembly 1600 that is interchangeably attached to the first 1500. The second assembly 1600 can be quickly disconnected from the first 1500 and replaced to replenish the supply of fastening means during a surgical procedure.

The first handle assembly 1500 includes a steering housing 1510, as depicted in FIG. 4. A steering sleeve 1520 surrounds the housing 1510 and is rotated to articulate the outer catheter 10. A steering sleeve lock 1530 is also included on an outer surface of the housing 1510 adjacent to the steering sleeve 1520. Disengagement of the lock 1530 allows free rotation of the steering sleeve 1520. Alternatively, the steering sleeve 1520 can be rotated without disengaging the lock 1530, producing an audible clicking sound and increased resistance to rotation of the steering sleeve 1520. A hemostasis port 1540 is also included within the steering housing 1510 and is attached to the proximal end of the outer catheter 10, allowing removal and reinsertion of inner catheters during exchange of the second handle assembly 1600.

The fastener module, or second handle assembly 1600, comprises a deployment housing 1610. A hemostasis port 1620, which is attached to an inner catheter 20 of the catheter assembly, is located within the housing 1610. Also within the housing 1610 and attached to the hemostasis port 1620 is an advancement slide 1630 which is actuated by an advancement sleeve 1640 surrounding the housing 1610. Adjacent to the advancement sleeve 1640 is an advancement sleeve lock 1650, which must be disengaged to rotate the advancement sleeve 1640. Re-engaging the lock 1650 will lock the advancement sleeve 1640 in position. The housing 1610 further includes a deployment slide 1670, which is coupled to the advancement slide 1630 by a deployment sleeve 1660 surrounding the deployment housing 1610. Because of the coupling, rotation of the advancement sleeve 1640 causes the two slides 1630, 1670 and the deployment sleeve 1660 to move in unison. This causes the inner catheter 20 and an optical fiber 30 having at least one fastening means positioned over, within, or in conjunction with it to move in unison during advancement as well. The deployment sleeve 1660 also has an associated tab 1680 on the outer surface of the housing 1610 for preventing rotation of the sleeve 1660 until, in one embodiment, at least 0.60 inches of advancement has occurred.

The fastener module 1600 may also include a deployment housing quick release button 1690, as shown in FIG. 4. By depressing the button 1690, the two handle assemblies 1500, 1600 can be disconnected. The second handle, assembly 1600 is pulled from the first 1500 until the inner catheter 20 is completely free of the first assembly's hemostasis port 1540. Reassembly of the two parts requires feeding the inner catheter 20 into the first assembly's hemostasis port 1540 until the deployment housing 1610 can be inserted into the steering housing 1510 and an audible click is heard. This indicates that the two housings are locked together. Locating tab and corresponding slot detailing may further be included on the two handle assemblies to prevent rotational misalignment of the inner and outer catheters during assembly. Any other method of locking the two handles together is also contemplated. A fiber position micro-adjustment 1695 may additionally be included within the deployment housing 1610 for adjustment of the distal tip of the optical fiber 30 relative to the distal tip of the inner catheter 20. The micro-adjustment component is available only during manufacturing to assist in assembly and alignment.

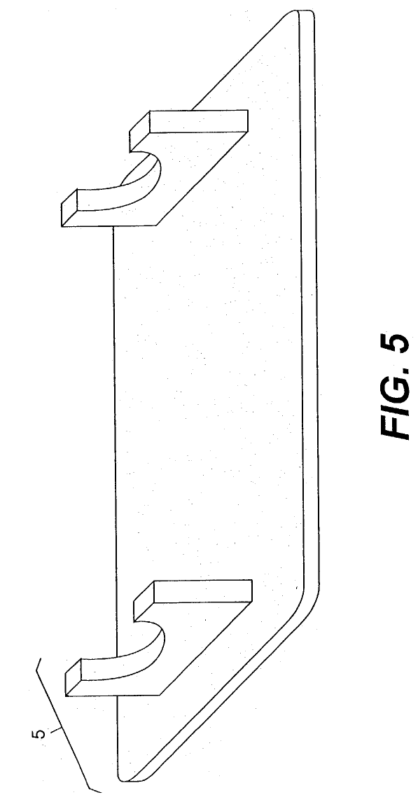

FIG. 5 illustrates a cradle 5 for use with embodiments of the catheter hand-piece apparatus 2, 3, 4. The catheter hand-piece can be stabilized on the cradle 5 during a surgical procedure. The cradle supports the catheter hand-piece in at least two locations and allows free rotation or incremental rotation during torquing of the handle.

While this invention has been described in conjunction with specific embodiments thereof, it is evident that many alternatives, modifications and variations will be apparent to those skilled in the art. Accordingly, the embodiments of the invention as set forth herein are intended to be illustrative, not limiting. Various changes may be made without departing from the spirit and scope of the invention as defined in the following claims.

What is claimed is:

1. A catheter hand-piece apparatus for controlling functions of a catheter during a surgical procedure, comprising:
a handle, which is connected to a catheter assembly; and
a fastener module, interchangeably located within said handle.

2. The catheter hand-piece apparatus according to claim 1, wherein said handle comprises:
a clear molded lid;
two enclosure sections between which said lid is pivotally attached;
a tubular axle located between said enclosure sections and attached thereto by a snap-ring; and
a nosepiece, adjustably mounted to said axle, which is attached to a catheter sheath of said catheter assembly.

3. The catheter hand-piece apparatus according to claim 2, wherein said nosepiece is spring-loaded.

4. The catheter hand-piece apparatus according to claim 2, wherein said nosepiece is fixed.

5. The catheter hand-piece apparatus according to claim 1, wherein said fastener module comprises:
an irrigation port;
a flexible sheath attached to said irrigation port and extending to a micro-adjustment boss component to which it is attached;
a tubular micro-adjustment knob slidably positioned about said flexible sheath and thread-assembled to said micro-adjustment boss component, said knob being interchangeably mounted to said handle;
an optical fiber, enabling transmission of laser energy to a surgical site, said optical fiber surrounded by a protective sheath, which extends from said irrigation port to a connector that is slidably positioned along the optical fiber;
a tubular fastener-pusher surrounding said optical fiber, extending from said irrigation port to a point abutting the most proximally positioned fastening means in communication with said optical fiber;
an internal o-ring located within said micro-adjustment boss component, which creates a fluid-tight seal between said o-ring and an outside surface of said fastener-pusher;
an external o-ring positioned about said fastener-pusher at the proximal end, which creates a fluid-tight seal between said external o-ring and said flexible sheath; and
at least one fastening means, which is loaded over said optical fiber, wherein said optical fiber is independently movable with respect to said fastening means.

6. The catheter hand-piece apparatus according to claim 5, wherein said fastener-pusher is comprised of tubing.

7. The catheter hand-piece apparatus according to claim 6, wherein said tubing is selected from the group consisting of polyimide, polytetrafluoroethylene, and fluorinated ethylenepropylene.

8. The catheter hand-piece apparatus according to claim 5, wherein said fastener-pusher is comprised of a wound coil.

9. The catheter hand-piece apparatus according to claim 5, wherein said fastening means is comprised of nitinol.

10. The catheter hand-piece apparatus according to claim 5, wherein said fastening means is comprised of stainless steel.

11. A catheter hand-piece apparatus for controlling functions of a catheter during a surgical procedure, comprising:
a handle, which is connected to a catheter assembly;
a fastener module, interchangeably located within said handle; and
a fastener module control mechanism, located within said handle, for advancing and withdrawing components of said fastener module.

12. The catheter hand-piece apparatus according to claim 11, wherein said handle comprises:
a clear molded lid;
two enclosure sections between which said lid is pivotally attached;
a tubular axle located between said enclosure sections and attached thereto by a snap-ring; and
a nosepiece, adjustably mounted to said axle, which is attached to a catheter sheath of said catheter assembly.

13. The catheter hand-piece apparatus according to claim 11, wherein said fastener module comprises:
an irrigation port;
a flexible sheath attached to said irrigation port and extending to a micro-adjustment boss component to which it is attached;
a tubular micro-adjustment knob slidably positioned about said flexible sheath and thread-assembled to said micro-adjustment boss component, said knob being interchangeably mounted to said handle;
an optical fiber, enabling transmission of laser energy to a surgical site, said optical fiber surrounded by a protective sheath, which extends from said irrigation port to a connector that is slidably positioned along the optical fiber;
a tubular fastener-pusher surrounding said optical fiber, extending from said irrigation port to a point abutting the most proximally positioned fastening means in communication with said optical fiber;
an internal o-ring located within said micro-adjustment boss component, which creates a fluid-tight seal between said o-ring and an outside surface of said fastener-pusher;
an external o-ring positioned about said fastener-pusher at the proximal end, which creates a fluid-tight seal between said external o-ring and said flexible sheath; and
at least one fastening means, which is loaded over said optical fiber, wherein said optical fiber is independently movable with respect to said fastening means.

14. The catheter hand-piece apparatus according to claim 12, wherein said fastener module control mechanism comprises:
an adjustment knob pair, externally positioned on opposite sides of said enclosure sections;
a geared shaft located within said handle, to which said knob pair is mounted, for propelling said fastener module distally via rack and pinion detailing;
a pair of advancing arms, the first of which is pivotally mounted to a carriage component, which is located within said molded enclosures, and slidably attached to said lid, the second of which is slidably located within said carriage component and between said enclosure sections, wherein said second arm associates with said geared shaft through rack and pinion detailing for advancing said fastener module.

15. The catheter hand-piece apparatus according to claim 14, wherein the proximal end of said second advancing arm works collaboratively with said micro-adjustment boss component, facilitating linear micro-adjustment of optical fiber positioning.

16. A catheter hand-piece apparatus for controlling functions of a catheter during a surgical procedure, comprising:
a handle, which is connected to a catheter assembly;
a fastener module, interchangeably located within said handle; and
an articulation control mechanism, located within said handle and attached to an indwelling adjustment filament, for adjusting the angular position of said catheter during a surgical procedure.

17. The catheter hand-piece apparatus according to claim 16, wherein said adjustment filament comprises a braided wire.

18. The catheter hand-piece apparatus according to claim 16, wherein said adjustment filament comprises a single wire.

19. The catheter hand-piece apparatus according to claim 16, wherein said adjustment filament comprises a monofilament.

20. The catheter hand-piece apparatus according to claim 16, wherein said handle comprises:
a clear molded lid;
two enclosure sections between which said lid is pivotally attached, a tubular axle located between said enclosures and attached thereto by a snap-ring; and
a nosepiece, adjustably mounted to said axle, which is attached to a catheter sheath of said catheter assembly.

21. The catheter hand-piece apparatus according to claim 16, wherein said fastener module comprises:
an irrigation port;
a flexible sheath attached to said irrigation port and extending to a micro-adjustment boss component to which it is attached;
a tubular micro-adjustment knob slidably positioned about said flexible sheath and thread-assembled to said micro-adjustment boss component, said knob being interchangeably mounted to said handle;
an optical fiber, enabling transmission of laser energy to a surgical site, said optical fiber surrounded by a protective sheath, which extends from said irrigation port to a connector that is slidably positioned along the optical fiber;
a tubular fastener-pusher surrounding said optical fiber, extending from said irrigation port to a point abutting the most proximally positioned fastening means in communication with said optical fiber;
an internal o-ring located within said micro-adjustment boss component, which creates a fluid-tight seal between said o-ring and an outside surface of said fastener-pusher;
an external o-ring positioned about said fastener-pusher at the proximal end, which creates a fluid-tight seal between said external o-ring and said flexible sheath; and
at least one fastening means, which is loaded over said optical fiber, wherein said optical fiber is independently movable with respect to said fastening means.

22. The catheter hand-piece apparatus according to claim 20, wherein said articulation control mechanism comprises:
an adjustment knob pair, externally positioned on opposite sides of said enclosure sections;
a gear component, to which said knob pair is mounted, located within said handle, for translating rotary motion of said knob pair into linear motion of a tubular mounting collar and to an associated boss through rack and pinion detailing, for pulling said adjustment filament attached to said boss.

23. A catheter hand-piece apparatus for controlling functions of a catheter during a surgical procedure, comprising:
a handle, which is connected to a catheter assembly;
a fastener module, interchangeably located within said handle; and
an apposition control mechanism, located within said handle, for advancing an inner sheath of a catheter delivery assembly thereby creating an appositional force between an outer sheath and a graft and vessel wall combination.

24. The catheter hand-piece apparatus according to claim 23, wherein said handle comprises:
a clear molded lid;
two enclosure sections between which said lid is pivotally attached;
a tubular axle located between said enclosure sections and attached thereto by a snap-ring; and
a nosepiece, adjustably mounted to said axle, which is attached to a catheter sheath of said catheter assembly.

25. The catheter hand-piece apparatus according to claim 23, wherein said fastener module comprises:
an irrigation port;
a flexible sheath attached to said irrigation port and extending to a micro-adjustment boss component to which it is attached;
a tubular micro-adjustment knob slidably positioned about said flexible sheath and thread-assembled to said micro-adjustment boss component, said knob being interchangeably mounted to said handle;
an optical fiber, enabling transmission of laser energy to a surgical site, said optical fiber surrounded by a protective sheath, which extends from said irrigation port to a connector that is slidably positioned along the optical fiber;
a tubular fastener-pusher surrounding said optical fiber, extending from said irrigation port to a point abutting the most proximally positioned fastening means in communication with said optical fiber;
an internal o-ring located within said micro-adjustment boss component, which creates a fluid-tight seal between said o-ring and an outside surface of said fastener-pusher;
an external o-ring positioned about said fastener-pusher at the proximal end, which creates a fluid-tight seal between said external o-ring and said flexible sheath; and
at least one fastening means, which is loaded over said optical fiber, wherein said optical fiber is independently movable with respect to said fastening means.

26. The catheter hand-piece apparatus according to claim 24, wherein said apposition control mechanism comprises:
an adjustment knob pair, externally positioned on opposite sides of said enclosure sections;
a gear component, to which said knob pair is mounted and located within said handle, for adjusting the angular positioning of a carriage component located within said handle, said carriage component being attached to an inner sheath of a catheter assembly, and through rack and pinion detailing advancing said inner sheath of the catheter assembly and creating an appositional force between said sheath and a graft and vessel wall combination.

27. A catheter hand-piece apparatus for controlling functions of a catheter during a surgical procedure, comprising:
a handle, which is connected to a catheter assembly;
a fastener module, interchangeably located within said handle;
a fastener module control mechanism, located within said handle, for advancing and withdrawing components of said fastener module;
an articulation control mechanism, located within said handle and attached to an indwelling adjustment filament, for adjusting the angular position of said catheter during a surgical procedure;
an apposition control mechanism, located within said handle, for advancing an inner sheath of a catheter delivery assembly thereby creating an appositional force between said inner sheath and a graft and vessel wall; and
a locking mechanism located within said handle, for selectively engaging said articulation or apposition control mechanisms.

28. The catheter hand-piece apparatus according to claim 27, wherein said handle comprises:
a clear molded lid;
two enclosure sections between which said lid is pivotally attached;
a tubular axle located between said enclosure sections and attached thereto by a snap-ring; and
a nosepiece, adjustably mounted to said axle, which is attached to a catheter sheath of said catheter assembly.

29. The catheter hand-piece apparatus according to claim 27, wherein said fastener module comprises:
an irrigation port;
a flexible sheath attached to said irrigation port and extending to a micro-adjustment boss component to which it is attached;
a tubular micro-adjustment knob slidably positioned about said flexible sheath and thread-assembled to said micro-adjustment boss component, said knob being interchangeably mounted to said handle;
an optical fiber, enabling transmission of laser energy to a surgical site, said optical fiber surrounded by a protective sheath, which extends from said irrigation port to a connector that is slidably positioned along the optical fiber;
a tubular fastener-pusher surrounding said optical fiber, extending from said irrigation port to a point abutting the most proximally positioned fastening means in communication with said optical fiber;
an internal o-ring located within said micro-adjustment boss component, which creates a fluid-tight seal between said o-ring and an outside surface of said fastener-pusher;
an external o-ring positioned about said fastener-pusher at the proximal end, which creates a fluid-tight seal between said external o-ring and said flexible sheath; and
at least one fastening means, which is loaded over said optical fiber, wherein said optical fiber is independently movable with respect to said fastening means.

30. The catheter hand-piece apparatus according to claim 27, wherein said locking mechanism comprises a spring-loaded assembly of components located within said handle, in which an axle mounted flexible yoke component works in collaboration with an activation button, for selectively engaging said articulation or apposition control mechanisms by communicating with each specific mechanism through ratchet detailing.

31. A catheter hand-piece apparatus for controlling functions of a catheter during a surgical procedure, comprising:
a first tubular handle, which is connected to a catheter assembly;
a second tubular handle, which engages with said first handle; and
a fastener module, located within said second handle.

32. The catheter hand-piece apparatus according to claim 31, wherein said first handle further comprises:
an articulation control mechanism, located within said handle and attached to an adjustment filament, for adjusting the angular position of said catheter during a surgical procedure;
an apposition control mechanism, located within said handle, for advancing an inner sheath of a catheter delivery assembly thereby creating an appositional force between an outer sheath and a graft and vessel wall combination;
an irrigation port of said handle for delivery of a flush solution around at least one fastening means.

33. The catheter hand-piece apparatus according to claim 32, wherein said articulation control mechanism comprises:
a knob, surrounding said handle; and
a gear component, located within said handle and to which said knob is mounted, for translating rotary motion into linear motion.

34. The catheter hand-piece apparatus according to claim 32, wherein said articulation control mechanism comprises:
a linear slide; and
a locking mechanism.

35. The catheter hand-piece apparatus according to claim 32, wherein said apposition control mechanism comprises:
a knob, surrounding said handle; and
a gear component, located within said handle and to which said knob is mounted, for translating rotary motion into linear motion.

36. The catheter hand-piece apparatus according to claim 32, wherein said apposition control mechanism comprises:
a linear slide; and
a locking mechanism.

37. The catheter hand-piece apparatus according to claim 31, wherein said fastener module further comprises:
a first knob surrounding said second handle;
a first gear component within said second handle and to which said first knob is mounted, for translating rotary into linear motion, thereby extending an optical fiber which is attached to said second handle and deploying at least one fastening means over, within, or in conjunction with said optical fiber;
a second knob surrounding said second handle; and
a second gear component located within said handle and to which said second knob is mounted, for translating rotary into linear motion, thereby adjusting the length of said optical fiber.

38. The catheter hand-piece apparatus according to claim 31, wherein said fastener module further comprises:
a first linear slide, for extending an optical fiber which is attached to said second handle and deploying at least one fastening means over said optical fiber;
a second linear slide, for adjusting the length of said optical fiber; and
two locking mechanisms, said first locking mechanism for securing a position of said first linear slide, and said second locking mechanism for securing a position of said second linear slide.

39. A catheter hand-piece apparatus for controlling functions of a catheter during a surgical procedure, comprising:
a tubular handle, which is connected to a catheter assembly;
an articulation control mechanism, located within said handle and attached to an adjustment filament, for adjusting the angular position of said catheter during a surgical procedure;
an apposition control mechanism, located within said handle, for advancing an inner sheath of a catheter delivery assembly thereby creating an appositional force between an outer sheath and a graft and vessel wall;
a fastener mechanism, located within said handle, for extending and adjusting the length of an optical fiber and deploying at least one fastening means over, within, or in conjunction with said optical fiber; and
an irrigation port of said handle for delivery of a flush solution around at least one fastening means.

40. The catheter hand-piece apparatus according to claim 39, wherein said articulation control mechanism comprises:
a knob surrounding said handle; and
a gear component, located within said handle and to which said knob is mounted, for translating rotary into linear motion.

41. The catheter hand-piece apparatus according to claim 39, wherein said articulation control mechanism comprises:
a linear slide; and
a locking mechanism.

42. The catheter hand-piece apparatus according to claim 39, wherein said apposition control mechanism comprises:
a knob surrounding said handle; and
a gear component, located within said handle and to which said knob is mounted, for translating rotary motion into linear motion.

43. The catheter hand-piece apparatus according to claim 39, wherein said apposition control mechanism comprises:
a linear slide; and
a locking mechanism.

44. The catheter hand-piece apparatus according to claim 39, wherein said fastener mechanism comprises:
a first knob and associated gear component, located within said handle, for translating rotary into linear motion, thereby extending an optical fiber which is attached to said handle and deploying at least one fastening means over said optical fiber; and
a second knob and associated gear component, located within said handle, for translating rotary into linear motion, thereby adjusting the length of said optical fiber during manufacturing.

45. The catheter hand-piece apparatus according to claim 39, wherein said fastener mechanism comprises:
a first linear slide, for extending an optical fiber which is attached to said handle and deploying at least one fastening means over said optical fiber;
a second linear slide, for adjusting the length of said optical fiber; and
a locking mechanism for securing a position of said linear slide.

46. A catheter hand-piece apparatus for controlling the functions of a catheter during a surgical procedure, comprising:
a first handle assembly, which is attached to an outer catheter of a catheter assembly; and
a fastener module, which is a second handle assembly interchangeably attached to said first handle assembly.

47. The catheter hand-piece apparatus according to claim 46, wherein said first handle assembly comprises:
a steering housing;
a steering sleeve surrounding said housing for articulating said outer catheter;
a steering sleeve lock, located on an outer surface of said housing adjacent to said steering sleeve, depression of which allows free rotation of said steering sleeve; and
a hemostasis port within said housing, which is attached to a proximal end of said outer catheter.

48. The catheter hand-piece apparatus according to claim 46, wherein said fastener module comprises:
a deployment housing;
a hemostasis port within said housing, which is attached to an inner catheter of said catheter assembly;
an advancement slide within said housing, which is attached to said hemostasis port;
an advancement sleeve surrounding said housing for actuating said advancement slide;
an advancement sleeve lock located on an outer surface of said housing adjacent to said advancement sleeve;
a deployment sleeve surrounding said housing, which couples a deployment slide to said advancement slide; and
a tab located on the outer surface of said housing adjacent to said deployment sleeve for preventing rotation of said deployment sleeve.

49. The fastener module according to claim 48, further comprising a deployment housing quick release tab.

50. The fastener module according to claim 48, further comprising a fiber position micro-adjustment within said housing.

51. The catheter hand-piece apparatus according to claim 46, further comprising at least one locating tab and corresponding slot on said handle assemblies.

52. The catheter hand-piece apparatus according to claims 31, 39, or 46 further comprising a cradle for stabilizing said apparatus during a surgical procedure.

53. A method of positioning a catheter assembly within a vessel in preparation for deployment of at least one fastening means at a surgical site, comprising the steps of:
controlling the advancement of said catheter assembly to a position adjacent said surgical site with a catheter hand-piece;
articulating a catheter tip of said catheter assembly by pulling an indwelling adjustment filament attached to an articulation control mechanism located within said catheter hand-piece;
advancing an inner sheath of said catheter assembly through an articulated catheter with an apposition control mechanism located within said hand-piece until the distal end of said inner sheath makes contact with a graft and which upon further advancement, forces said catheter assembly into contact with a vessel wall directly opposite said surgical site; and
locking said inner sheath in a position directly opposite said surgical site with a locking mechanism.

54. The method according to claim 53, further comprising the step of rotating a catheter sheath of said catheter assembly independent of said catheter hand-piece apparatus, as needed.

55. The method according to claim 53, further comprising the step of adjusting the radial positioning of said inner sheath's distal tip with a nose-piece connected to said catheter hand-piece.

56. A method of controlling deployment of a fastening means to a surgical site and determining a quantity of fastening means deployed with a catheter hand-piece apparatus, comprising the steps of:
creating an aperture in a graft and vessel combination;
advancing an optical fiber, at least one fastening means, and a fastener-pusher with a fastener module control mechanism located within said catheter hand-piece;
irrigating contact surfaces between said optical fiber and said at least one fastening means, and between said at least one fastening means and the internal surface of an inner sheath of a catheter assembly through an irrigation port located within or remotely from said catheter hand-piece;
withdrawing said optical fiber independently of said at least one fastening means and said fastener-pusher with said fastener module control mechanism, thereby deploying at least one fastening means; and
indicating the quantity of fastening means deployed or remaining in said fastener module within said catheter hand-piece.

57. The method according to claim 56, wherein the step of creating an aperture in a graft and vessel wall combination comprises activating or terminating a laser energy transmission from a remote controller.

58. The method according to claim 56, wherein the step of irrigating contact surfaces comprises irrigating said surfaces immediately following each graft and tissue ablation.

59. The method according to claim 56, further comprising the step of adjusting the axial positioning of the tip of said optical fiber with respect to a distal face of an over-fiber positioned fastening means, which is most distally located, with said catheter hand-piece apparatus.

60. A method of repositioning a catheter assembly and replenishing at least one fastening module during a surgical procedure for repeated deployment of fastening means, comprising the steps of:
withdrawing an inner sheath of said catheter assembly and an optical fiber with a catheter hand-piece apparatus;
unlocking an articulated catheter tip and returning said tip to a collinear alignment with respect to said catheter assembly with said catheter hand-piece apparatus;
repeatedly repositioning said catheter assembly to facilitate further deployment of fastening means with said catheter hand-piece apparatus; and
replenishing a supply of fastening means to a surgical site mid-procedure by removing the spent fastener module and replacing the module with another fastener module having at least one fastening means therein.

61. The method according to claim 60, further comprising the step of unlocking said inner sheath of said catheter assembly prior to withdrawal.

62. A method of positioning a catheter assembly within a vessel in preparation for deployment of at least one fastening means at a surgical site, controlling deployment of said fastening means to said surgical site, and determining a quantity of fastening means deployed with a catheter hand-piece apparatus, comprising the steps of:
controlling the advancement of said catheter assembly to a position adjacent said surgical site with said catheter hand-piece;
articulating a catheter tip of said catheter assembly by pulling an indwelling adjustment filament attached to an articulation control mechanism located within said catheter hand-piece,
advancing an inner sheath of said catheter assembly through an articulated catheter with an apposition control mechanism located within said hand-piece until the distal end of said inner sheath makes contact with a graft and which upon further advancement, forces said catheter assembly into contact with a vessel wall directly opposite said surgical site;
locking said inner sheath in a position directly opposite said surgical site with a locking mechanism;
creating an aperture in a graft and vessel combination;
advancing said optical fiber, at least one fastening means, and a fastener-pusher with a fastener module control mechanism located within said catheter hand-piece;
irrigating contact surfaces between said optical fiber and said at least one fastening means, and between said at least one fastening means and the inner surface of said inner sheath through an irrigation port located within or remotely from said catheter hand-piece;
withdrawing said optical fiber independently of said at least one fastening means and said fastener-pusher with said fastener module control mechanism, thereby deploying at least one fastening means; and
indicating the quantity of fastening means deployed or remaining in said fastener module within said catheter hand-piece.

63. A method of positioning and repositioning a catheter assembly within a vessel in preparation for deployment of at least one fastening means at a surgical site and replenishing at least one fastening module during a surgical procedure for repeated deployment of fastening means, comprising the steps of:
controlling the advancement of said catheter assembly to a position adjacent said surgical site with a catheter hand-piece;
articulating a catheter tip of said catheter assembly by pulling an indwelling adjustment filament attached to an articulation control mechanism located within said catheter hand-piece;
advancing an inner sheath of said catheter assembly through an articulated catheter with an apposition control mechanism located within said hand-piece until the distal end of said inner sheath makes contact with a graft and which upon further advancement, forces said catheter assembly into contact with a vessel wall directly opposite said surgical site;
locking said inner sheath in a position directly opposite said surgical site with a locking mechanism;
withdrawing said inner sheath and an optical fiber with said catheter hand-piece apparatus;
unlocking said articulated catheter tip and returning said tip to a collinear alignment with said catheter assembly with said catheter hand-piece apparatus;
repeatedly repositioning said catheter assembly to facilitate further deployment of fastening means with said catheter hand-piece apparatus; and
replenishing a supply of fastening means to said surgical site mid-procedure by removing the spent fastener module and replacing the module with another fastener module having at least one fastening means therein.

64. A method of positioning and repositioning a catheter assembly within a vessel in preparation for deployment of at least one fastening means at a surgical site, controlling deployment of said at least one fastening means at said surgical site, displaying the quantity of fastening means deployed, and replenishing at least one fastening module during a surgical procedure for repeated deployment of fastening means with a catheter hand-piece apparatus, comprising the steps of:
controlling the advancement of said catheter assembly to a position adjacent said surgical site;
articulating a catheter tip of said catheter assembly by pulling an indwelling adjustment filament attached to an articulation control mechanism located within said catheter hand-piece;
advancing an inner sheath of said catheter assembly with an apposition control mechanism located within said hand-piece until the distal end of said inner sheath makes contact with a graft and which upon further advancement, forces said catheter assembly into contact with a vessel wall directly opposite said surgical site;
locking said inner sheath in a position directly opposite said surgical site with a locking mechanism;
creating an aperture in a graft and vessel combination;
advancing said optical fiber, at least one fastening means, and a fastener-pusher with a fastener module control mechanism located within said catheter hand-piece;
irrigating contact surfaces between said optical fiber and said at least one fastening means, and between said at least one fastening means and the inner surface of said inner sheath through an irrigation port located within or remotely from said catheter hand-piece;
withdrawing said optical fiber independently of said at least one fastening means and said fastener-pusher with said fastener module control mechanism, thereby deploying at least one fastening means;
indicating the quantity of fastening means deployed or remaining in said fastener module within said catheter hand-piece;
withdrawing said inner sheath and said optical fiber with said catheter hand-piece apparatus;
unlocking said articulated catheter tip and returning said tip to a collinear alignment with respect to said catheter assembly with said catheter hand-piece apparatus;
repeatedly repositioning said catheter assembly to facilitate further deployment of fastening means with said catheter hand-piece apparatus; and
replenishing a supply of fastening means to said surgical site mid-procedure by removing the spent fastener module and replacing the module with another fastener module having at least one fastening means therein.
